(19) United States
(12) Patent Application Publication
(10) Pub. No.: US 2026/0251058 A1
(43) Pub. Date: Aug. 27, 2026

(54) WELL LOGGING APPARATUS WITH BUOYANCY ADJUSTMENT

(71) Applicant: Schlumberger Technology Corporation, Sugar Land, TX (US)

(72) Inventors: Cedric Perrin, Clamart (FR); Mehdi Hizem, Sugar Land, TX (US); Jose Luis Lopes, Clamart (FR); Florian Becker, Clamart (FR)

(21) Appl. No.: 19/550,537

(22) Filed: Feb. 26, 2026

Related U.S. Application Data
(60) Provisional application No. 63/769,279, filed on Mar. 10, 2025.
(60) Provisional application No. 63/763,357, filed on Feb. 26, 2025.

(51) Int. Cl. E21B 47/085; E21B 47/04; E21B 47/06; G01B 5/12; G01N 9/26; G01N 33/28; G01V 1/50

(52) U.S. Cl. CPC E21B 47/085; E21B 47/04; E21B 47/06; G01B 5/12; G01N 9/26; G01N 33/2823; G01V 1/50

(57) ABSTRACT

An untethered well logging apparatus includes a body, a buoyancy adjusting mechanism disposed in the body and configured to increase a buoyancy of the untethered well logging apparatus when a predetermined condition is met, and a downhole sensor disposed with the body.

### CROSS-REFERENCE TO RELATED APPLICATIONS

This application claims priority to U.S. Provisional Application No. 63/763,357, titled DEVICE AND METHOD FOR MULTI FINGER CALIPER MEASUREMENT, filed 26 February 2025 and claims priority to U.S. Provisional Application No. 63/769,279, titled DEVICE AND METHOD FOR MULTI FINGER CALIPER MEASUREMENT, filed 10 March 2025, both of which are hereby incorporated by reference in their entirety.

### BACKGROUND

Producing hydrocarbons from a wellbore drilled into a geological region involves measurements from downhole well-logging tools that are conveyed into the wellbore, where the measurements may be used to infer properties or characteristics of the geological region surrounding the wellbore, including the internal diameter of casing or tubing installed within the wellbore. Conventional well logging operations typically employ wireline or slickline conveyance systems to deploy measurement tools downhole, with caliper measurements being performed during logging operations to assess the condition of tubulars, detect corrosion, erosion, wear, and other defects, and to measure accumulation of scale or other deposits on internal surfaces. What is needed in the art are simple, cheap, and reliable downhole sensors and tools that require minimal oversight and control.

### SUMMARY

According to an aspect of the present disclosure, an untethered well logging apparatus is provided. The untethered well logging apparatus includes a body. The untethered well logging apparatus includes a buoyancy adjusting mechanism disposed in the body and configured to increase a buoyancy of the untethered well logging apparatus when a predetermined condition is met. The untethered well logging apparatus includes a downhole sensor disposed with the body.

According to other aspects of the present disclosure, the untethered well logging apparatus may include one or more of the following features. The downhole sensor may include a depth sensor. The downhole sensor may include at least one of a magnetometer, accelerometer, or gyro. The downhole sensor may include a low friction caliper device attached to the body and configured to determine a distance between the body and an internal wall of a well. The low friction caliper device may include a sensing arm configured to articulate relative to the body, a spring urging the sensing arm radially outward relative to the body, a sensor configured to determine an angle between the sensing arm and the body, and a roller provided at a distal end of the sensing arm and configured to roll on the internal wall of the well. The untethered well logging apparatus may further include an actuator configured to retract the sensing arm relative to the body. The body may include a notch configured to receive the roller of the sensing arm when the sensing arm is retracted. The low friction caliper device may include a sensing finger configured to extend radially outward relative to the body, a spring urging the sensing finger radially outward relative to the body, a sensor arrangement configured to determine an outward radial position of the sensing finger, and a bow spring having a central portion attached to a distal end of the sensing finger. The bow spring may include a first end and a second end slidably attached to the body. The low friction caliper device may include a piezoelectric sensor configured to generate an ultrasound wave and receive an ultrasound echo reflected by the internal wall. The piezoelectric sensor may be convex and configured to generate a pseudo-spherical ultrasound wave. The downhole sensor may include a fluid density sensor. The fluid density sensor may include a differential pressure sensor. The low friction caliper device may include a sphere configured to freely rotate and to move relative to the body, an urging element urging the sphere radially outward relative to the body, and a sensor assembly configured to determine a position of the sphere relative to the body.

According to another aspect of the present disclosure, a well logging apparatus is provided. The well logging apparatus includes a body having a main axis and an outer surface. The well logging apparatus includes a buoyancy adjusting mechanism configured to increase a buoyancy of the untethered well logging apparatus when a predetermined condition is met. The well logging apparatus includes a low friction caliper device provided on the outer surface of the body and configured to determine a distance between the body and an internal wall of a well.

According to other aspects of the present disclosure, the well logging apparatus may include one or more of the following features. The low friction caliper device may include a sensing arm configured to articulate relative to the body, a spring urging the sensing arm radially outward relative to the body, a sensor configured to determine an angle between the sensing arm and the body, and a roller provided at a distal end of the sensing arm and configured to roll on the internal wall of the well. Activating the buoyancy adjusting mechanism may include a gas tank configured to release a compressed gas into a ballast tank to displace a liquid from the ballast tank.

According to another aspect of the present disclosure, an untethered well logging apparatus is provided. The untethered well logging apparatus includes a body having a main axis and an outer surface. The untethered well logging apparatus includes a ballast tank having an inlet port provided in an upper portion of the ballast tank and an outlet port provided in a lower portion of the ballast tank, the outlet port being connected to an outside environment. The untethered well logging apparatus includes a high-pressure tank configured to store ballast gas in a compressed state. The untethered well logging apparatus includes a valve connecting the high-pressure tank with the inlet port of the ballast tank. The untethered well logging apparatus includes a caliper device provided on the outer surface of the body and configured to determine a distance between the body and an internal wall of a well.

According to other aspects of the present disclosure, the untethered well logging apparatus may include one or more of the following features. The caliper device may include a sensing arm articulated with the body in a pivotable fashion, a spring urging the sensing arm towards an outward direction, and a roller provided at a distal end of the sensing arm and configured to roll on the internal wall of the well. The caliper device may include a piezoelectric sensor configured to generate an ultrasound wave and to receive an ultrasound echo reflected by the internal wall of the well. The caliper device may further include a differential pressure sensor configured to measure fluid density for determining sound velocity.

### BRIEF DESCRIPTION OF FIGURES

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1A illustrates a sensor device positioned within a wellbore, according to aspects of the present disclosure;

FIG. 1B illustrates a sensor device positioned within a wellbore alongside a plot of various corresponding sensor measurements, according to aspects of the present disclosure;

FIG. 1C illustrates a sensor device positioned within a wellbore alongside a plot of various corresponding sensor measurements, according to aspects of the present disclosure;

FIG. 2 illustrates a sensor device showing a ballast mechanism, according to aspects of the present disclosure;

FIG. 3 illustrates a caliper assembly, according to aspects of the present disclosure;

FIG. 4A illustrates a sensor device with a caliper assembly, according to aspects of the present disclosure;

FIG. 4B illustrates the sensor device of FIG. 4A with caliper arms in a various retracted configurations, according to aspects of the present disclosure;

FIG. 5A illustrates an isometric view of a sensor device in a descent configuration, according to aspects of the present disclosure;

FIG. 5B illustrates a cross-sectional view of a sensor device configured for logging up operations, according to aspects of the present disclosure;

FIG. 5C illustrates a cross-sectional view of the sensor device of FIG. 5B in an ascension configuration, according to aspects of the present disclosure;

FIG. 5D illustrates a cross-sectional view of a release mechanism in an activated state, according to aspects of the present disclosure;

FIG. 6A illustrates a cross-sectional view of a depth sensor configuration, according to aspects of the present disclosure;

FIG. 6B illustrates a horizontal cross-sectional view of a sensor device with multiple depth sensors, according to aspects of the present disclosure;

FIG. 7 illustrates a cross-sectional view of a sensor device with a bow spring caliper assembly, according to aspects of the present disclosure;

FIG. 8A illustrates a cross-sectional view of a sensor device configured for ultrasonic caliper measurement, according to aspects of the present disclosure;

FIG. 8B illustrates a horizontal cross-sectional view of the sensor device of FIG. 8A, according to aspects of the present disclosure;

FIG. 9A illustrates a cross-sectional view of a caliper assembly with a rotating sphere, according to aspects of the present disclosure; and

FIG. 9B illustrates a top view of a sensor device with multiple caliper assemblies, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to an untethered well logging apparatus configured to perform downhole measurements within a wellbore. In some cases, the untethered well logging apparatus may be conveyed through the wellbore without a wireline or slickline connection to the surface. The untethered well logging apparatus may descend through the wellbore using gravitational force acting on the apparatus and may ascend through the wellbore using buoyancy force.

In some embodiments, the untethered well logging apparatus may include a buoyancy adjusting mechanism configured to modify the buoyancy of the apparatus when a predetermined condition is met. The buoyancy adjusting mechanism may increase the buoyancy of the untethered well logging apparatus to facilitate ascent through the wellbore after the apparatus has reached a target depth. The predetermined condition may include reaching a specified depth, reaching a specified time after deployment, or receiving a trigger signal.

The untethered well logging apparatus may include one or more downhole sensors configured to acquire measurements during conveyance through the wellbore. In some cases, the downhole sensors may include a caliper device configured to determine a distance between the apparatus and an internal wall of the wellbore. The caliper device may be a low friction caliper device configured to reduce dragging force opposing motion of the apparatus through the wellbore. The low friction caliper device may enable caliper measurements to be performed during descent, ascent, or both descent and ascent of the apparatus through the wellbore.

In some cases, the untethered well logging apparatus may include a depth sensor configured to determine a position of the apparatus within the wellbore. Other sensor assemblies may include at least one of a magnetometer, accelerometer, or gyro. The depth sensor may enable correlation of caliper measurements with depth within the wellbore. The untethered well logging apparatus may further include a controller configured to manage data acquisition and processing from the downhole sensors.

The untethered well logging apparatus may provide advantages for well logging operations by eliminating the need for wireline or slickline conveyance equipment. In some cases, the untethered well logging apparatus may reduce operational complexity and may enable measurements in wells where wireline access is limited or unavailable.

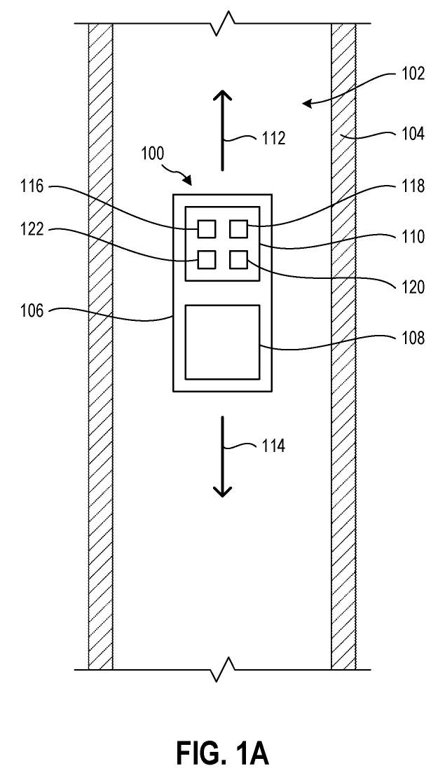

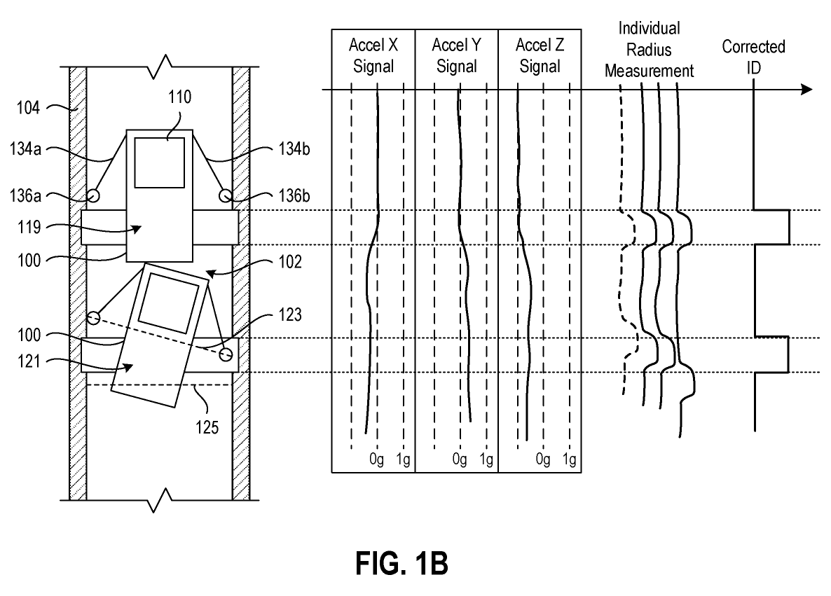

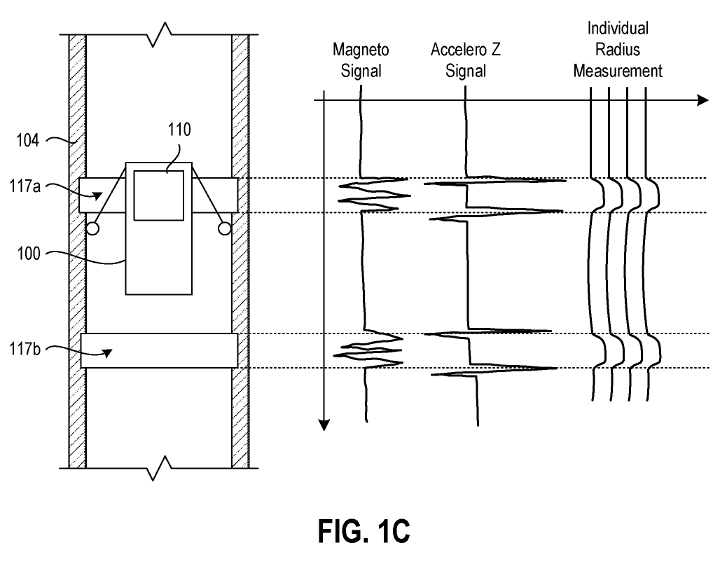

Referring to FIG. 1A, a sensor device 100 may be positioned within a wellbore 102. The wellbore 102 may extend through a casing 104. In some embodiments, the tools and structure described herein may also be used to measure and log in an uncased borehole for direct measurements of a formation through which the borehole extends. The sensor device 100 may be an untethered well logging apparatus configured to travel through the wellbore 102 without a wireline or slickline connection to the surface.

The sensor device 100 may include a body 106. The body 106 may house various internal components of the sensor device 100. A ballast 108 may be disposed in the body 106. The ballast 108 may function as a buoyancy adjusting mechanism configured to increase a buoyancy of the sensor device 100 when a predetermined condition is met. The predetermined condition may include reaching a target depth within the wellbore 102, reaching a specified time after deployment, or receiving a trigger signal.

The sensor device 100 may further include a sensor assembly 110 positioned within the body 106. The sensor assembly 110 may include one or more downhole sensors disposed with the body 106. The sensor assembly 110 may include a depth sensor 116 configured to determine a position of the sensor device 100 within the wellbore 102. In some embodiments, the sensor assembly 110 may include at least one of the depth sensor 116, magnetometer, accelerometer, or gyro. In at least one embodiment, the depth sensor 116 may utilize 3-axis accelerometers combined with 3-axis gyros to determine positioning of the sensor device 100 in real-time within the wellbore 102.

As noted above, the sensor assembly 110 may include an accelerometer, as shown in FIG. 1B. The sensor device 100 in FIG. 1B is shown within the wellbore 102 defined by the casing 104 in a first position 119 and the same sensor device 100 in the wellbore 102 in a second position 121. The sensor device 100 may include first and second arms 134a and 134b with respective rollers 136a and 136b contacting an inner surface of the casing 104. The sensor device 100 may include one or more sensors for measuring a distance of the opposing rollers 136a and 136b for determining an inner diameter of the casing 104 at any given position (e.g., 119 and 121).

As shown in FIG. 1B, in the second position 121, the sensor device 100 may temporarily be tilted at an angle such that the measured distance 123 between the rollers 136a and 136b is longer than the actual inner diameter 125 of the casing 104. Real time measurements from the accelerometer may be used to correct the measured distance 123 such that the measured distance 123 in combination with the measurements of the tilt angle of the sensor device 100 can be used to determine the corrected (i.e., actual) inner diameter 125 of the casing 104.

To the right of the illustration of the sensor device 100 shown in the wellbore 102 in FIG. 1B is a plot illustrating the X, Y, and Z measurements/reading from the three-axis accelerometer of the sensor assembly 110 corresponding to vertical positions within the casing 104 shown on the left. As seen in the plot on the right, the variations in the X, Y, and Z readings from the accelerometer over the vertical distance and time may indicate a tilt angle and orientation of the sensor device 100 within the wellbore 102 for diameter corrections and other measurement corrections. In addition, the plot shows that variations in the diameter of the casing 104, for example variations due to joint features and profiles, may be detected and corrected for as the sensor device 100 travels through the casing 104.

Also, in at least some embodiments, the sensor device 100 may include three axis magnetometers configured to identify casing joints, for example casing joints 117a and 117b shown in FIG. 1B. The sensor device 100 travels through the casing 104 and, as the sensor device 100 passes the joints 117a and 117b (and other joints throughout the casing 104), the magnetometer of the sensor assembly 110 may enable correlation of caliper measurements with a tally of the sections of the casing 104 joined together at the joints (e.g., 117a and 117b). This tally may be correlated to depth measurements to determine a depth of the sensor tool 100 in the casing 104.

The plot on the right in FIG. 1C illustrates the magnetometer and accelerometer readings along the length of the casing, as well as individual radius measurements from the sensor tool 100. As illustrated, signals from readings of the magnetometer and accelerometer Z-signal may correspond in position with the casing joints 117a and 117b. In addition, in at least one embodiment, the sensor assembly 110 of the untethered sensor device 100 may include three axis gyros, to identify velocity and apparatus rotation while logging.

The sensor assembly 110 may also include a caliper sensor 118. The caliper sensor 118 may be a low friction caliper device attached to the body 106 and configured to determine a distance between the body 106 and an internal wall of the wellbore 102. The sensor assembly 110 may further include another sensor 120 configured to acquire additional measurements within the wellbore 102. Additional measurements may include wellbore fluid characteristics, composition, tool wear measurements, wellbore casing imaging, or monitoring, and so forth.

A controller 122 may be operatively connected to the depth sensor 116, the caliper sensor 118, and the other sensor 120. The controller 122 may include processing capabilities for executing a logic programmed sequence. The controller 122 may manage an acquisition chain for the sensors of the sensor assembly 110. The controller 122 may further include a means of recording data acquired by the sensor assembly 110. The controller 122 may also include a means of communicating with other electronic devices when the sensor device 100 is outside the wellbore 102, such as for downloading an acquisition program or uploading recorded sensor data.

Referring again to FIG. 1A, an upward direction 112 may be indicated toward the surface of the wellbore 102, and a downward direction 114 may be indicated toward a bottom of the wellbore 102. The sensor device 100 may be configured to travel within the wellbore 102 in both the upward direction 112 and the downward direction 114. The sensor device 100 may descend through the wellbore 102 in the downward direction 114 using gravitational force acting on the sensor device 100. The ballast 108 may enable buoyancy adjustment to facilitate movement of the sensor device 100 in the upward direction 112 through the wellbore 102 after the predetermined condition is met.

Any of the features, components, and/or parts, including the arrangements and configurations thereof shown in FIGS. 1A through 1C can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 1A through 1C.

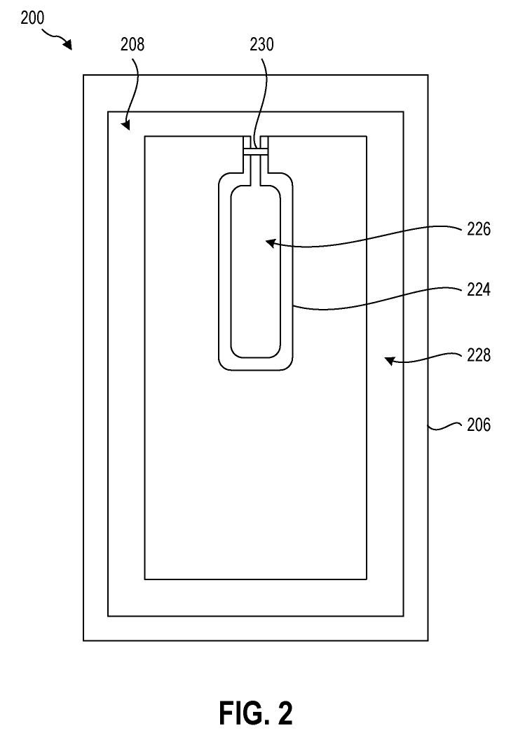

Referring to FIG. 2, a sensor device 200 may include a body 206 having a main axis and an outer surface. The body 206 may house various internal components of the sensor device 200. A ballast 208 may be disposed in the body 206. The ballast 208 may function as a buoyancy adjusting mechanism configured to increase a buoyancy of the sensor device 200 when a predetermined condition is met.

The ballast 208 may include a tank 224 configured to store high pressure gas 226 in a compressed state. The tank 224 may function as a high-pressure tank configured to store ballast gas in the compressed state. The high-pressure gas 226 may be compressed to a pressure that is increased by at least the same proportion compared to an expected downhole pressure as a ratio of a ballast volume versus a tank volume.

The ballast 208 may further include a ballast tank 228 positioned below the tank 224. The ballast tank 228 may have an inlet port provided in an upper portion of the ballast tank 228 and an outlet port provided in a lower portion of the ballast tank 228. The outlet port may be connected to an outside environment. The ballast tank 228 may be filled with liquid at the surface to facilitate sinking of the sensor device 200 into a well. The liquid may be well liquid or another suitable fluid.

A valve 230 may connect the tank 224 with the inlet port of the ballast tank 228. The valve 230 may be positioned at the upper portion of the ballast tank 228 and may control release of the high-pressure gas 226 from the tank 224 into the ballast tank 228. The valve 230 may be configured to release a compressed gas into the ballast tank 228 to displace a liquid from the ballast tank 228.

When the predetermined condition is met, the valve 230 may be opened to release the high-pressure gas 226 into the ballast tank 228. The valve 230 can be mechanically activated or a one-shot opening orifice based on explosive detonation, electrically activated (example: exowasher). The high-pressure gas 226 may expand into the ballast tank 228, displacing liquid contained therein through the outlet port. The displacement of liquid may reduce an overall density of the sensor device 200 by replacing liquid mass with gas. The reduction in density may increase the buoyancy of the sensor device 200, allowing the sensor device 200 to ascend within the wellbore in the upward direction.

The expansion of the high-pressure gas 226 may be governed by the ideal gas law. As the sensor device 200 ascends through the wellbore, the pressure may decrease and the high-pressure gas 226 may continue expansion into the ballast tank 228. Excess gas may continue escaping from the outlet port at the lower portion of the ballast tank 228 in an autoregulating manner, even as temperature decreases during ascent. The autoregulating gas expansion may maintain buoyancy of the sensor device 200 throughout the ascent to the surface.

In some embodiments, the buoyancy adjusting mechanism may include a releasable weight configured to be detached from the sensor device when the predetermined condition is met. The releasable weight may be secured to the body using a securing latch that can be actuated by an electric release mechanism. When the electric release mechanism is triggered, the securing latch may disengage, allowing the releasable weight to separate from the sensor device. The separation of the releasable weight may reduce the overall mass of the sensor device while maintaining the same volume, thereby increasing the buoyancy of the sensor device. The electric release mechanism may be triggered by a controller based on reaching a target depth, reaching a specified time after deployment, reaching a specific pressure, counting a specific number of casing/tubing joints based on tally or receiving a trigger signal.

In some embodiments, the releasable weight may include a dissolvable material configured to dissolve in wellbore fluid over a predetermined time period. The dissolvable material may be selected to dissolve at a rate that corresponds to an expected descent time of the sensor device to a target depth. As the dissolvable material dissolves, the mass of the sensor device may decrease, eventually causing the buoyancy of the sensor device to exceed the gravitational force acting on the sensor device. The use of dissolvable material may eliminate the need for active electronic triggering mechanisms, providing a passive buoyancy adjustment approach. In other cases, the buoyancy adjusting mechanism may combine multiple approaches, such as a gas-based ballast system in combination with a releasable weight, to provide redundancy and ensure reliable ascent of the sensor device to the surface.

Any of the features, components, and/or parts, including the arrangements and configurations thereof shown in FIG. 2 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 2.

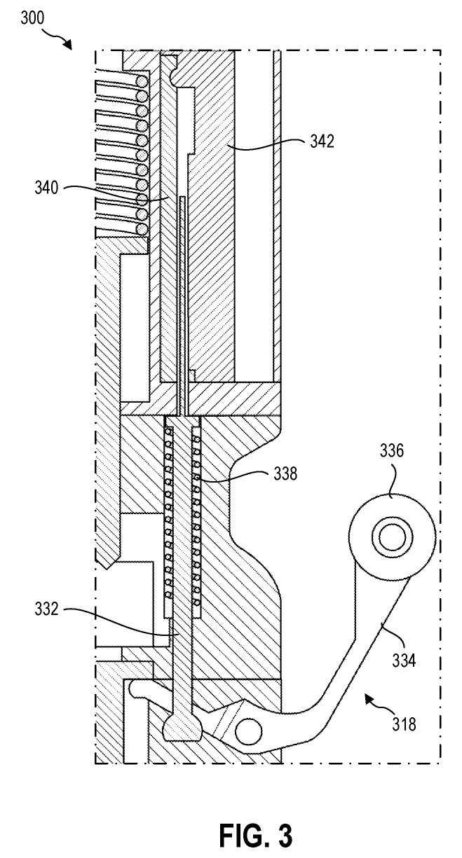

Referring to FIG. 3, a caliper assembly 318 may be provided on an outer surface of the body and may be configured to determine a distance between the body and an internal wall of a wellbore or casing or other tubular in the wellbore. The caliper assembly 318 may function as a low friction caliper device attached to the body. The caliper assembly 318 may include several components arranged to measure an internal diameter of a wellbore or pipe.

The caliper assembly 318 may include a rod 332 extending vertically through the assembly. The rod 332 may be connected to an arm 334. The arm 334 may function as a sensing arm configured to articulate relative to the body. The arm 334 may be articulated with the body in a pivotable fashion. The arm 334 may extend outward from the body and may pivot about a pivot point to accommodate variations in the internal diameter of the wellbore.

A spring 338 may be positioned within the caliper assembly 318. The spring 338 may urge the rod 332 downward and thus the arm 334 radially outward relative to the body, thereby articulating the arm 334 about the pivot point. The spring 338 may urge the arm 334 radially outward relative to the body to maintain contact between the arm 334 and the internal wall of the well. The spring 338 may urge the arm 334 towards an outward direction to ensure continuous engagement with the wellbore wall during measurement operations.

A roller 336 may be provided at a distal end of the arm 334. The roller 336 may be configured to roll on the internal wall of the well. The roller 336 may reduce friction at a pipe interface during measurement operations. The roller 336 may minimize dragging force opposing motion of the sensor device through the wellbore. The rolling action of the roller 336 may enable the caliper assembly 318 to function as a low friction caliper device suitable for use in an untethered well logging apparatus that relies on buoyancy for conveyance.

A displacement sensor 340 may be positioned within the caliper assembly 318. The displacement sensor 340 may function as a sensor configured to determine an angle between the arm 334 and the body. The displacement sensor 340 may determine a position of the arm 334 relative to a main axis of the body. The displacement sensor 340 may translate radial displacement of the arm 334 to linear motion for measurement purposes.

In at least one embodiment, the displacement sensor 340 may be of resistive nature, such as a potentiometer. In at least one embodiment, the displacement sensor 340 may be of electromagnetic nature, such as a Linear Variable Differential Transformer (LVDT) or inductance. In at least one embodiment, the displacement sensor 340 may be of capacitive nature. In at least one embodiment, the displacement sensor 340 may be of strain-based nature. In at least one embodiment, the displacement sensor 340 may include a Differential Variable Reluctance Transducer (DVRT) that translates radial displacement to linear motion.

Electronics 342 may be located in an upper portion of the caliper assembly 318. The electronics 342 may be coupled to, and process signals from, the displacement sensor 340. The electronics 342 may convert an analog electric signal from the displacement sensor 340 to a digital signal. The digital signal may be conveyed either to the surface or to downhole memory for storage and subsequent retrieval.

The caliper assembly 318 may further include an actuator configured to retract the arm 334 relative to the body. The actuator may enable the arm 334 to be stowed during descent or ascent operations when measurement is not being performed. The body may include a notch configured to receive the roller 336 of the arm 334 when the arm 334 is retracted. The notch may allow the caliper assembly 318 to be stowed in a compact configuration, reducing the overall profile of the sensor device during conveyance through the wellbore.

Any of the features, components, and/or parts, including the arrangements and configurations thereof shown in FIG. 3 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 3.

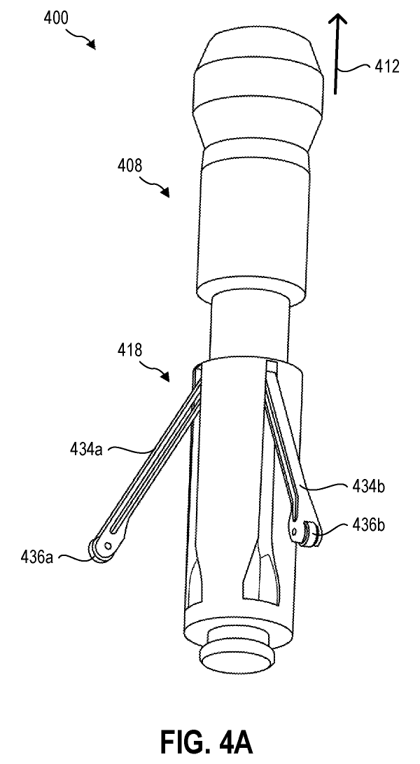

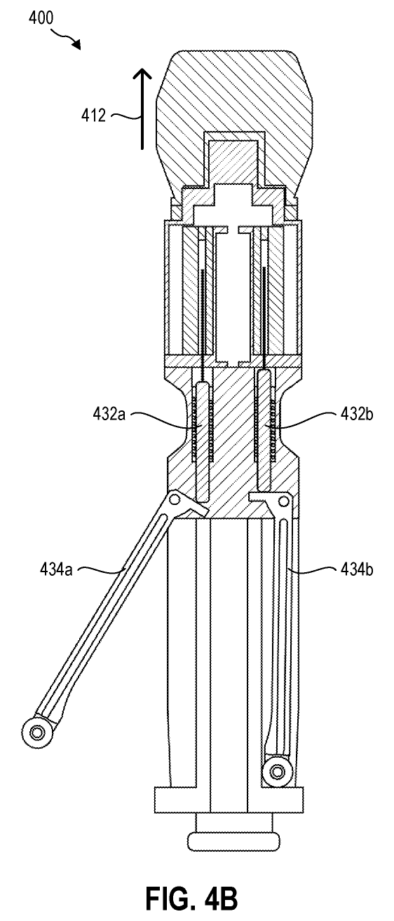

Referring to FIG. 4A, a sensor device 400 may include a ballast 408 positioned in an upper portion of the sensor device 400. An upward direction 412 may be indicated, showing the orientation of the sensor device 400 during operation within a wellbore. The sensor device 400 may include a caliper assembly 418 provided on an outer surface of the body. The caliper assembly 418 may be configured to determine a distance between the body and an internal wall of a well.

The caliper assembly 418 may include a first arm 434a and a second arm 434b that extend outwardly from the body of the sensor device 400. The first arm 434a may be provided at a distal end with a first roller 436a, and the second arm 434b may be provided at a distal end with a second roller 436b. The first roller 436a and the second roller 436b may be configured to roll on the internal wall of the well. The first roller 436a and the second roller 436b may reduce friction at a pipe interface and may minimize dragging force opposing motion of the sensor device 400 through the wellbore.

As shown in FIG. 4A, the first arm 434a and the second arm 434b are shown in an extended position, enabling the caliper assembly 418 to contact and measure the internal diameter of the wellbore during logging operations. In at least one embodiment, the caliper assembly 418 may include at least four arms distributed around a circumference of the body for sensing a pipe internal diameter. In at least one embodiment, the caliper assembly 418 may include up to eight arms distributed around the circumference of the body for sensing the pipe internal diameter. The caliper assembly 418 may be configured to measure internal diameter up to 7 inches with accuracy of +/-0.02 inches.

Referring to FIG. 4B, the sensor device 400 is shown in a configuration where caliper arms are in different states for illustrative purposes. The sensor device 400 may include a body oriented along a main axis with the upward direction 412 indicated at a top of the figure. The sensor device 400 may include a first rod 432a and a second rod 432b positioned on opposite sides of the body. The first rod 432a and the second rod 432b may be displacement sensing components that translate radial displacement to linear motion for measurement purposes.

The first rod 432a may be connected to the first arm 434a, and the second rod 432b may be connected to the second arm 434b. As shown in FIG. 4B, the first arm 434a is shown in an extended configuration projecting outward from the body, while the second arm 434b is shown in a retracted configuration along the body of the sensor device 400. In the retracted configuration, the second arm 434b may be folded inward against the body, which may reduce an overall profile of the sensor device 400. The retracted state may be utilized during descent of the sensor device 400 through the wellbore or during ascent after measurement operations have been completed. The extended state of the first arm 434a may enable caliper measurement while the sensor device 400 travels through the wellbore.

Any of the features, components, and/or parts, including the arrangements and configurations thereof shown in FIGS. 4A and FIG. 4B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 4A and FIG. 4B.

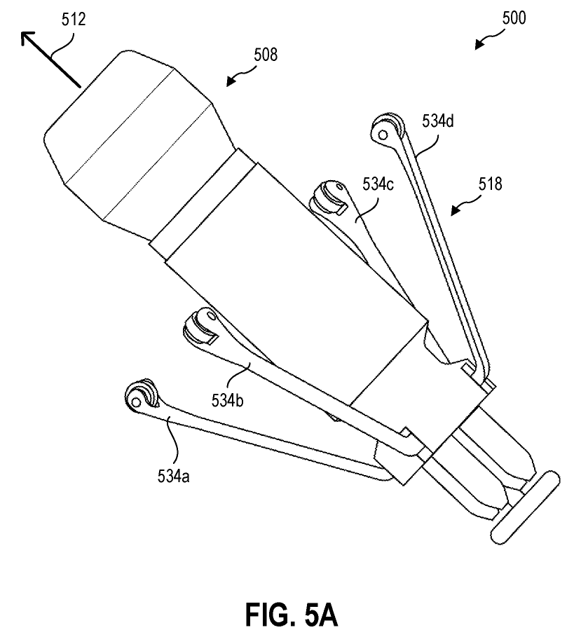

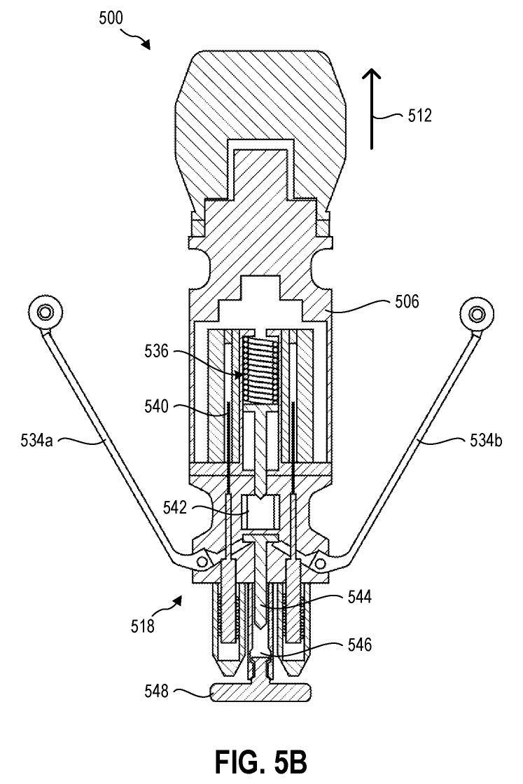

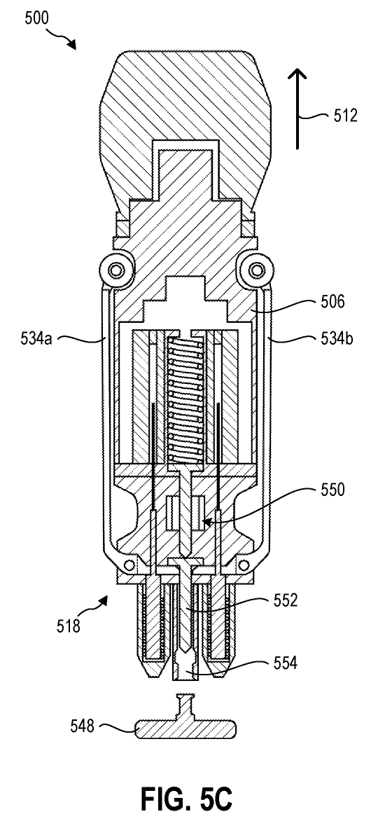

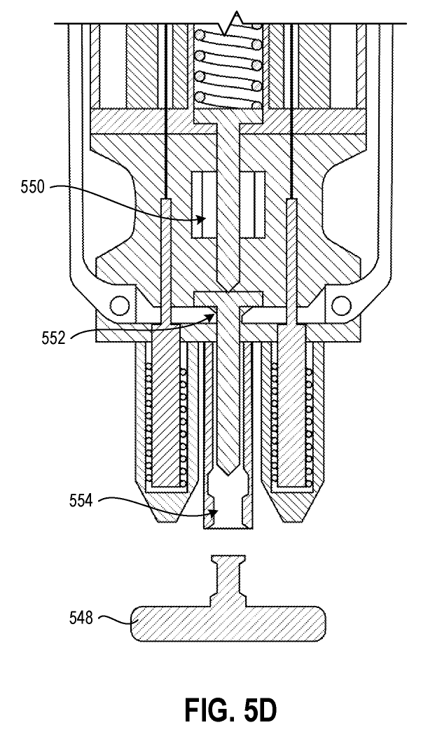

Referring to FIG. 5A, a sensor device 500 is shown in a descent configuration. The sensor device 500 may include a body 580 having a main longitudinal axis indicated along directional arrow 512 and an outer surface. A ballast 508 may be positioned at an upper portion of the sensor device 500. The ballast 508 may facilitate descent of the sensor device 500 through a wellbore. An upward direction 512 is indicated, showing the orientation of the sensor device 500 within the well.

The sensor device 500 may include a caliper assembly 518 provided on the outer surface of the body 580. The caliper assembly 518 may include a plurality of arms distributed around a circumference of the body 580. As shown in FIG. 5A, the caliper assembly 518 may include a first arm 534a, a second arm 534b, a third arm 534c, and a fourth arm 534d. The first arm 534a, the second arm 534b, the third arm 534c, and the fourth arm 534d is shown in an open or extended position, projecting outward from the body 580 in a radial direction. Each of the first arm 534a, the second arm 534b, the third arm 534c, and the fourth arm 534d may be articulated with the body 580 in a pivotable fashion, allowing the arms to move between open and closed positions. Distal ends of the first arm 534a, the second arm 534b, the third arm 534c, and the fourth arm 534d may be configured to contact an internal wall of a well to determine a distance between the body 580 and the internal wall. The arrangement of the four arms around a perimeter of the body 580 may enable multi-finger caliper measurement of a pipe internal diameter.

Referring to FIG. 5B, the sensor device 500 may be configured for logging up operations. The sensor device 500 may include a body 506 oriented along the upward direction 512. The sensor device 500 may incorporate the caliper assembly 518 positioned along a lower portion of the body 506. The caliper assembly 518 may include the first arm 534a and the second arm 534b extending outward from the body 506. As shown in FIG. 5B, the first arm 534a and the second arm 534b are shown in an extended configuration relative to the body 506.

A spring-loaded release mechanism 536 may be positioned in the body 506. The spring-loaded release mechanism 536 may be configured to actuate movement of the first arm 534a and the second arm 534b. A displacement sensor 540 may be provided to measure a radial position of the first arm 534a and the second arm 534b relative to the body 506.

An electric release mechanism 542 may be configured to initiate a release sequence when a predetermined condition is met. In at least some embodiments, the condition may include at least one of pressure, temperature, depth, casing joint, or velocity. For example, if the sensor device 500 is not moving for a determined time, this may indicate the tool is stuck. If such an example, the release mechanism 536 may be actuated to free the sensor device 500 and allow its ascension.

In at least one embodiment, the electric release mechanism 542 may include an electrically controlled release device (ECRD) where electrical power supplied to the electric release mechanism 542 generates localized heat that melts a soldered compound to release a locking mechanism. In at least one embodiment, the electric release mechanism 542 may include an exowasher where electrical power supplied to the electric release mechanism 542 generates localized heat that melts a soldered compound to release the locking mechanism. Once the soldered compound is molten, mechanical properties of the locking mechanism may be reduced, and the locking mechanism may release a constrained body in two to four parts that allow additional mechanisms to activate.

A securing latch 544 may be positioned below the electric release mechanism 542. The securing latch 544 may function to lock the first arm 534a and the second arm 534b in a closed position during ascension of the sensor device 500. A latch 546 may be provided to release a dissolvable or releasable material 548 from the sensor device 500. The dissolvable or releasable material 548 may serve as a releasable weight that, when detached, reduces an overall weight of the sensor device 500 and allows the sensor device 500 to ascend through the well using buoyancy. In some embodiments, the material 548 may be dissolvable such that the material 548 does not to present a problem in a future life of the well.

The arrangement of components within the body 506 may enable the sensor device 500 to descend through the well with the first arm 534a and the second arm 534b extended for measurement. Upon firing of the electric release mechanism 542, the spring-loaded release mechanism 536 may actuate the securing latch 544 to retract and lock the first arm 534a and the second arm 534b while simultaneously releasing the dissolvable material 548 to facilitate ascension.

Referring to FIG. 5C, the sensor device 500 is shown in an ascension configuration. The sensor device 500 may include the body 506 oriented along the upward direction 512. The caliper assembly 518 may include the first arm 534a and the second arm 534b positioned on opposite sides of the body 506. The first arm 534a and the second arm 534b are shown in a retracted position along the body 506.

A fired release 550 may represent a state of the electric release mechanism 542 after activation. An activated securing latch 552 is shown engaged within the body 506. The activated securing latch 552 may function to maintain the first arm 534a and the second arm 534b in a retracted position during ascension of the sensor device 500. A released latch 554 may be depicted in a disengaged state, indicating that the dissolvable material 548 has been released from the sensor device 500.

Referring to FIG. 5D, a close-up view of the mechanisms shown in FIG. 5C are shown. As shown, the sensor device 500 is in an activated state after the electric release mechanism 542 has been triggered. The fired release 550 is shown in an upper portion of the assembly, indicating that the electric release mechanism 542 has been actuated. Below the fired release 550, the activated securing latch 552 is visible, demonstrating an engaged position after being triggered by the spring-loaded release mechanism 536. The released latch 554 is shown in a lower portion of the assembly, illustrating a state of the latch 546 after the latch 546 has been disengaged to release the dissolvable material 548. The dissolvable material 548 may be depicted at a bottom of the figure, shown in a released configuration separate from a main body of the sensor device 500.

Once the electric release mechanism 542 is fired, an upper piston may be free to move. Under spring force from the spring-loaded release mechanism 536, the upper piston may move down and actuate the securing latch 544. Once the securing latch 544 is activated, the securing latch 544 may release the dissolvable material 548 and lock the first arm 534a and the second arm 534b in the closed position.

In at least one embodiment, dimensions of the caliper assembly 518 may be optimized to reduce an assembly overall length and increase measurement accuracy down to +/-0.01 inches for 4-inch internal diameter pipe measurement.

Any of the features, components, and/or parts, including the arrangements and configurations thereof shown in FIGS. 5A through 5D can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 5A through 5D.

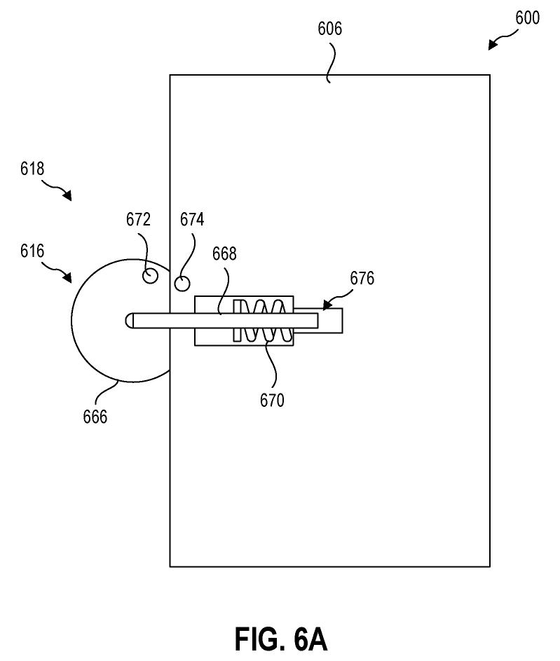

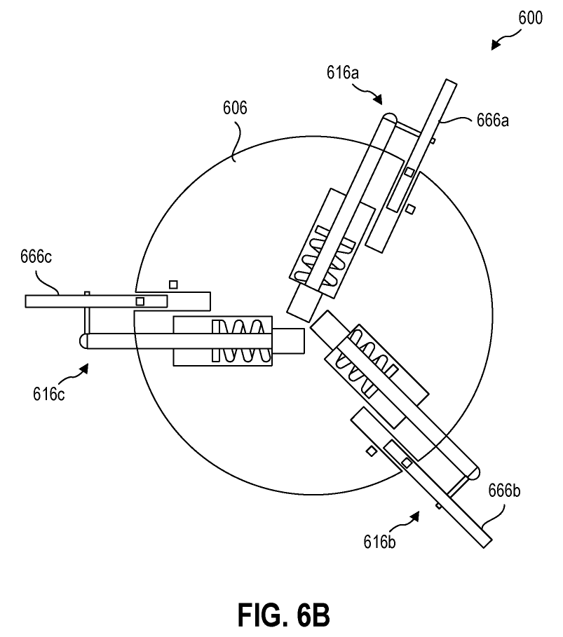

Referring to FIG. 6A, a sensor device 600 may include a body 606 positioned adjacent to a well. The sensor device 600 may include a depth sensor 616 configured to determine a position of the sensor device 600 within the well. The depth sensor 616 may also function as a caliper assembly 618 configured to determine a distance between the body 606 and an internal wall of the well.

The depth sensor 616 may include a wheel 666 mounted on the body 606. The wheel 666 may be configured to contact the internal wall of the well. The wheel 666 may be mounted on a suspension that is normal to a tubing wall and a travel direction of the sensor device 600. A rod 668 may extend from the body 606 and may support the wheel 666. The rod 668 may be configured to translate radial displacement of the wheel 666 to linear motion for measurement purposes.

A spring 670 may be provided to urge the wheel 666 in an outward direction toward the internal wall of the well. The spring 670 may maintain contact between the wheel 666 and the internal surface of the well during movement of the sensor device 600 through the well. The spring 670 and the rod 668 may maintain contact of the wheel 666 against the internal wall of the well as the sensor device 600 travels through the well.

A displacement sensor 676 may be provided to determine a radial position of the wheel 666 relative to the body 606. The displacement sensor 676 may measure displacement of the wheel 666 via the rod 668. The displacement sensor 676 may enable the wheel 666 and the rod 668 assembly to function as a caliper tool by measuring the radial position of the wheel 666, which corresponds to the distance between the body 606 and the internal wall of the well.

The depth sensor 616 may further include a magnet 672 associated with the wheel 666 and a Hall effect sensor 674 positioned to detect rotation of the magnet 672 as the wheel 666 rotates during movement of the sensor device 600 through the well. The wheel 666 may rotate along the internal surface of the well as the sensor device 600 travels through the well. The rotation of the wheel 666 detected by the Hall effect sensor 674 in conjunction with the magnet 672 may enable measurement of a distance traveled by the sensor device 600 within the well. Knowing a radius of the wheel 666, the traveled distance along the well may be extracted directly from the rotation count of the wheel 666 without ambiguity. The magnet 672 and the Hall effect sensor 674 may provide the distance traveled by the wheel 666 with known dimensions and may therefore serve as the depth sensor 616.

In at least one embodiment, the wheel 666 may include at least two magnets with one magnet having North facing up and another magnet having South facing up. In at least one embodiment, additional magnets may be added by interleaving orientations of the magnets. In at least one embodiment, a coil may be inserted in the body 606 in lieu of the Hall effect sensor 674. The coil may be terminated by a diode bridge, a capacitor, and a voltage regulator. The wheel 666 with magnets having alternating North and South orientations and the coil in the body 606 may function as an alternator for generating electrical energy to power the sensor device 600. The changes of electrical current orientation in the coil may also be used to measure depth.

In at least one embodiment, wheel rotation detection may be performed using a tachometer. In at least one embodiment, wheel rotation detection may be performed using optical detection with a hole and light. In at least one embodiment, wheel rotation detection may be performed using change in material property methods.

Referring to FIG. 6B, the sensor device 600 may include the body 606 having a circular cross-section. Distributed around a circumference of the body 606 may be three depth sensor assemblies positioned at angular intervals. A first depth sensor 616a may be associated with a first wheel 666a, a second depth sensor 616b may be associated with a second wheel 666b, and a third depth sensor 616c may be associated with a third wheel 666c. Each of the first wheel 666a, the second wheel 666b, and the third wheel 666c may be mounted on an arm extending radially outward from the body 606, with the wheels configured to contact the internal wall of the well.

The three-wheel arrangement may provide centering of the sensor device 600 within the well. Having more than one wheel, the sensor device 600 may detect and compensate for wheel slippage. Going to three wheels, the sensor device 600 may be centered within the borehole, tubing, or casing. The system with the first depth sensor 616a, the second depth sensor 616b, and the third depth sensor 616c about the body 606 may help maintain a central positioning of the body 606 within the well.

Each of the first depth sensor 616a, the second depth sensor 616b, and the third depth sensor 616c may be positioned to monitor rotation of a corresponding wheel, allowing the traveled distance along the well to be determined based on wheel radius and rotation count. The radial positioning of the first wheel 666a, the second wheel 666b, and the third wheel 666c may also enable caliper measurement functionality by detecting displacement of the arms relative to the body 606.

Any of the features, components, and/or parts, including the arrangements and configurations thereof shown in FIGS. 6A and FIG. 6B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 6A and FIG. 6B.

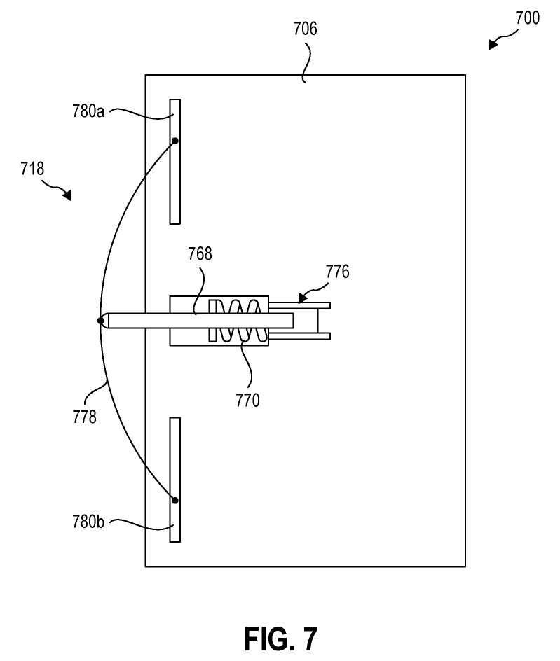

Referring to FIG. 7, a sensor device 700 may include a body 706 having a main axis and an outer surface. The sensor device 700 may include a caliper assembly 718 provided on the outer surface of the body 706. The caliper assembly 718 may be configured to determine a distance between the body 706 and an internal wall of a well. The caliper assembly 718 may function as a low friction caliper device attached to the body 706.

The caliper assembly 718 may include a rod 768 that extends in a radial direction from the body 706. The rod 768 may function as a sensing finger configured to extend radially outward relative to the body 706. The rod 768 may be configured to move in translation along the radial direction to accommodate variations in the internal diameter of a wellbore.

A spring 770 may be positioned within the caliper assembly 718. The spring 770 may urge the rod 768 toward an outward direction away from the body 706. The spring 770 may urge the rod 768 radially outward relative to the body 706 to maintain contact between the caliper assembly 718 and the internal wall of the well during measurement operations.

A displacement sensor 776 may be provided within the caliper assembly 718. The displacement sensor 776 may function as a sensor arrangement configured to determine an outward radial position of the rod 768. The displacement sensor 776 may determine a position of the rod 768 along the radial direction, which corresponds to the distance between the body 706 and the internal wall of the well.

The caliper assembly 718 may further include a bow spring 778. The bow spring 778 may have a central or intermediate portion attached to a distal end of the rod 768. The bow spring 778 may be configured to contact the internal wall of the well and to flex in response to variations in the internal diameter of the wellbore. The bow spring 778 may distribute contact force along a length of the bow spring 778, reducing localized friction at the interface with the wellbore wall.

The bow spring 778 may have two ends, with a first anchor 780a positioned at an upper portion of the body 706 and a second anchor 780b positioned at a lower portion of the body 706. The first end of the bow spring 778 may be slidably secured to the first anchor 780a, and the second end of the bow spring 778 may be slidably secured to the second anchor 780b. The first and second anchors 780a and 780b, respectively, may include slots or channels in which the opposing ends of the bow spring 778 may travel. The slidable attachment of the first end and the second end of the bow spring 778 to the body 706 via the first anchor 780a and the second anchor 780b may permit the bow spring 778 to flex and accommodate variations in the internal diameter of the well while maintaining contact with the well wall.

Any of the features, components, and/or parts, including the arrangements and configurations thereof shown in FIG. 7 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 7.

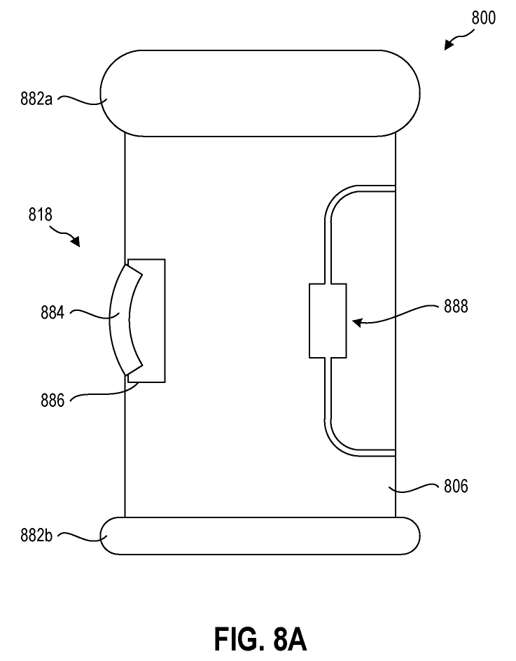

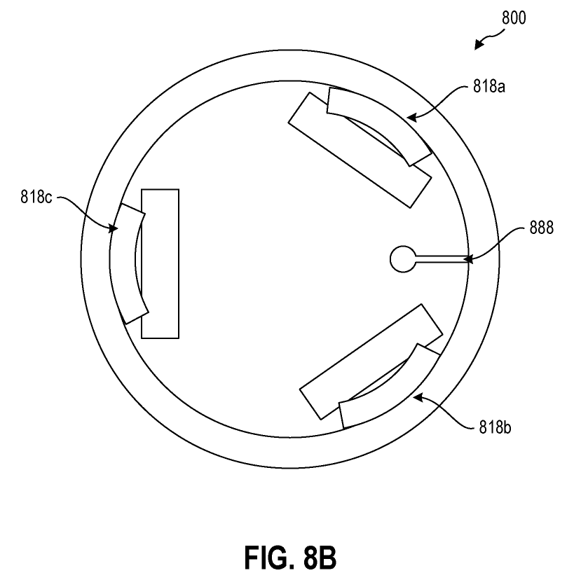

Referring to FIG. 8A, a sensor device 800 may be configured for ultrasonic caliper measurement. The sensor device 800 may include a body 806, which may house various internal components of the sensor device 800. A caliper assembly 818 may be provided on the body 806 and may be configured to determine a distance between the body 806 and an internal wall of a well. The caliper assembly 818 may function as a low friction caliper device attached to the body 806.

The sensor device 800 may include a first guide 882a positioned at an upper portion of the body 806 and a second guide 882b positioned at a lower portion of the body 806. The first guide 882a and the second guide 882b may be configured to maintain a central position of the body 806 within a wellbore. The first guide 882a and the second guide 882b may guide travel of the sensor device 800 through the wellbore. The first guide 882a and the second guide 882b may limit effects of eccentricity and tilt of the sensor device 800 within a tubing or borehole.

The caliper assembly 818 may include a piezoelectric sensor 884 provided on the body 806. The piezoelectric sensor 884 may be configured to generate an ultrasound wave and to receive an ultrasound echo reflected by the internal wall of the well. A backing 886 may be positioned behind the piezoelectric sensor 884. The backing 886 may provide structural support for the piezoelectric sensor 884 and may absorb rearward-directed acoustic energy. In at least one embodiment, the piezoelectric sensor 884 may be convex in shape. The convex shape of the piezoelectric sensor 884 may be configured to generate a pseudo-spherical ultrasound wave when combined with an electronic drive circuit. The pseudo-spherical ultrasound wave generated by the convex piezoelectric sensor 884 may compensate for apparatus eccentricity within the tubing or borehole. The pseudo-spherical ultrasound wave may also compensate for vertical tilt of the sensor device 800 versus a tubing axis. The piezoelectric sensor 884 combined with the first guide 882a and the second guide 882b may limit effects of eccentricity and tilt during ultrasonic caliper measurement.

A differential pressure sensor 888 may also be mounted on the body 806. The differential pressure sensor 888 may function as a fluid density sensor configured to measure fluid density. The differential pressure sensor 888 may include a vertical arrangement to provide measurements over a constant vertical height, which may provide fluid density. The differential pressure sensor 888 may be configured to measure fluid density for determining sound velocity.

An ultrasonic travel time of the ultrasound echo may be measured with reference to an internal clock of the sensor device 800. The travel time may be converted to distance by using sound velocity either as a predefined input or by using the fluid density measured by the differential pressure sensor 888. The fluid density measured by the differential pressure sensor 888 may be used with user input as a means of fluid typing. As an example, a user may set a fluid composition to be solved for to be oil and water, with predefined density for each. A measured density of a mixture may be used to extract a composition of the mixture in terms of oil and water. From predefined characteristics of the oil and water, an effective medium sound velocity may be calculated for use by the piezoelectric sensor 884.

The caliper assembly 818 may be configured to measure a second echo from an external pipe interface. Knowing sound velocity in metal, tubing thickness may be deduced from the second echo. Comparing the deduced tubing thickness to an original thickness, an estimation of metal loss may be extracted.

Referring to FIG. 8B, the sensor device 800 is shown in a horizontal cross-sectional view. The sensor device 800 may include a first caliper assembly 818a, a second caliper assembly 818b, and a third caliper assembly 818c distributed around a perimeter of the body 806. The first caliper assembly 818a, the second caliper assembly 818b, and the third caliper assembly 818c may be positioned at angular offsets around a circumference of the body 806 to provide multi-point measurement capability. In at least one embodiment, the first caliper assembly 818a, the second caliper assembly 818b, and the third caliper assembly 818c may be distributed at 120 degree offset angles around the circumference of the body 806.

Each of the first caliper assembly 818a, the second caliper assembly 818b, and the third caliper assembly 818c may include a piezoelectric sensor with backing material. The arrangement of the first caliper assembly 818a, the second caliper assembly 818b, and the third caliper assembly 818c around the circumference may enable the sensor device 800 to obtain azimuthal caliper measurements of the internal diameter of a wellbore or tubing. More sensors may be placed around the circumference to add resolution to the azimuthal measurement.

As shown in FIG. 8B, the differential pressure sensor 888 may also be positioned on the body 806 of the sensor device 800. The differential pressure sensor 888 may provide fluid density measurements for determining sound velocity used by the first caliper assembly 818a, the second caliper assembly 818b, and the third caliper assembly 818c for converting ultrasonic travel time to distance values.

Any of the features, components, and/or parts, including the arrangements and configurations thereof shown in FIGS. 8A and FIG. 8B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 8A and FIG. 8B.

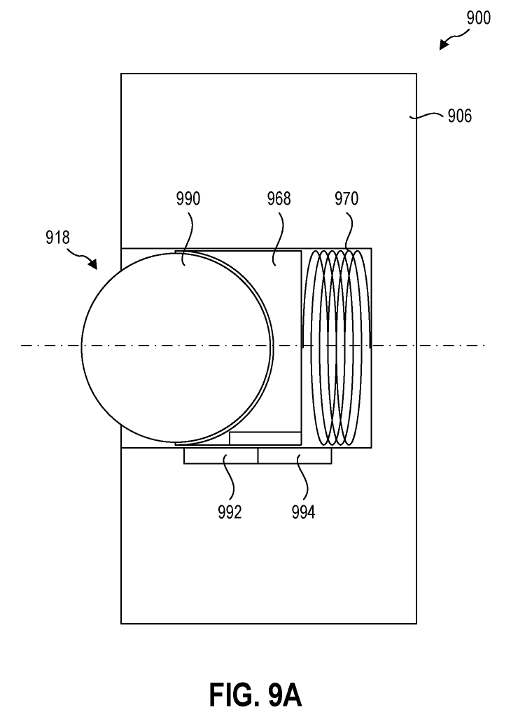

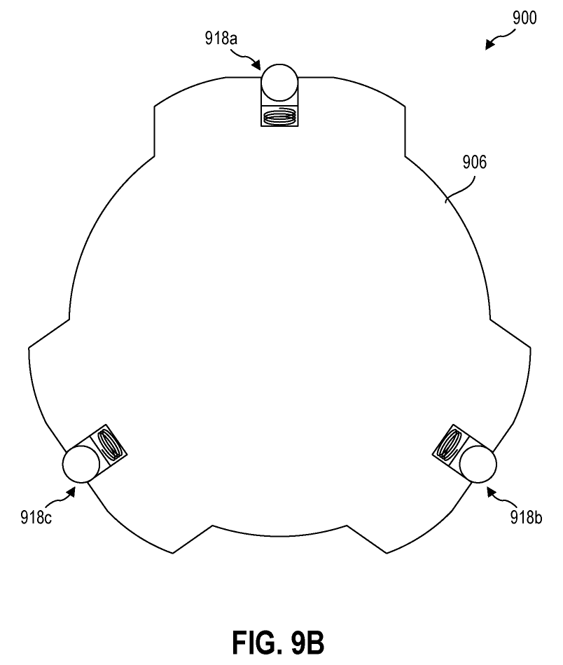

Referring to FIG. 9A, a sensor device 900 may include a body 906 having a main axis and an outer surface. The sensor device 900 may include a caliper assembly 918 provided on the outer surface of the body 906. The caliper assembly 918 may be configured to determine a distance between the body 906 and an internal wall of a well. The caliper assembly 918 may function as a low friction caliper device attached to the body 906.

The caliper assembly 918 may include a rotating sphere 990 accommodated within a cavity of the body 906. The rotating sphere 990 may be configured to freely rotate and to move relative to the body 906. The rotating sphere 990 may be able to rotate freely in any direction within the cavity, allowing both rotary-drilling and axially sliding motion. The rotating sphere 990 may move within the cavity along a radial axis to accommodate variations in the internal diameter of a wellbore. The rotating sphere 990 may be formed of an appropriately hard material suitable for contact with the internal wall of the well.

A spring 970 may be provided within the caliper assembly 918. The spring 970 may function as an urging element urging the rotating sphere 990 radially outward relative to the body 906. The spring 970 may push the rotating sphere 990 outward along the radial axis to maintain contact between the rotating sphere 990 and the internal wall of the well during measurement operations. In at least one embodiment, the rotating sphere 990 may be supported by a combination of the spring 970 and a pressure differential between internal mud pressure and downhole pressure. The pressure differential may provide a dampening effect that minimizes bearing bouncing during active drilling operations. The pressure differential may disappear while tripping out of hole and combining the spring 970 with the pressure differential approach may provide a preferred solution.

A retaining ring 978 may be installed on a face of the body 906 at an opening of the cavity. The retaining ring 978 may retain the rotating sphere 990 within the cavity while allowing the rotating sphere 990 to contact the internal wall of the well. A range of standoff measurement may be determined based on a radius of the rotating sphere 990. A maximum standoff may be reached when the rotating sphere 990 is barely kept in the cavity by the retaining ring 978, with the maximum being slightly smaller than the radius of the rotating sphere 990.

The caliper assembly 918 may further include a sensor assembly configured to determine a position of the rotating sphere 990 relative to the body 906. A rod 968 may be positioned adjacent to the rotating sphere 990 within the cavity. The rod 968 may function as a ferrite rod for positioning of the rotating sphere 990. A sidewall of the cavity may accommodate a recess aligned with a cylindrical axis of the cavity. The recess may house a linear-position sensor that directly provides the position of the rotating sphere 990.

A first coil 992 and a second coil 994 may be positioned to sense a position of the ferrite rod 968. The first coil 992 and the second coil 994 may work in conjunction with the rod 968 to form the sensor assembly configured to determine the position of the rotating sphere 990 relative to the body 906. The first coil 992 and the second coil 994 may detect changes in magnetic field as the rotating sphere 990 moves along the radial axis. In at least one embodiment, the sensor assembly may include a force gauge at a base of the cavity to determine compression of the spring 970. Hooke's law may then determine a length of the compressed spring 970 and hence the position of the rotating sphere 990.

In at least one embodiment, the sensor assembly may include a ferromagnetic material embedded in a bearing supporting the rotating sphere 990, for example embedded int he rod 968. The ferromagnetic material may be protected from the environment by a hard coating that prevents the ferromagnetic material from being chemically attacked by a corrosive environment and mechanically worn out, which may affect dimensions and magnetic state.

In at least one embodiment, the sensor assembly may include a measure pin extending from the bearing downward into a thinner hole at a bottom of the cavity. The thinner hole may house a linear-position sensor, and the measure pin may directly provide the position of the rotating sphere 990.

Referring to FIG. 9B, the sensor device 900 is shown in a top view. The sensor device 900 may include the body 906 having a generally circular cross-section with multiple protrusions extending outward from a perimeter. Disposed around a circumference of the body 906 may be a first caliper assembly 918a, a second caliper assembly 918b, and a third caliper assembly 918c. The first caliper assembly 918a may be positioned at an upper portion of the body 906, while the second caliper assembly 918b and the third caliper assembly 918c may be positioned at lower portions of the body 906 on opposite sides.

Each of the first caliper assembly 918a, the second caliper assembly 918b, and the third caliper assembly 918c may include a rotating sphere visible at an outer surface, which may be configured to contact the internal wall of the well during operation. The first caliper assembly 918a, the second caliper assembly 918b, and the third caliper assembly 918c may be distributed around the perimeter of the body 906 at offset angles to provide multi-point standoff measurements for determining the distance between the body 906 and the internal wall of the well. The arrangement of the first caliper assembly 918a, the second caliper assembly 918b, and the third caliper assembly 918c may enable the sensor device 900 to obtain caliper measurements from multiple azimuthal positions simultaneously. Combined standoff values from the first caliper assembly 918a, the second caliper assembly 918b, and the third caliper assembly 918c may provide both a borehole profile and a direct measurement of tool eccentricity.

Any of the features, components, and/or parts, including the arrangements and configurations thereof shown in FIGS. 9A and FIG. 9B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 9A and FIG. 9B.

The embodiments of downhole tools have been primarily described with reference to wellbore drilling operations; the downhole tools described herein may be used in applications other than the drilling of a wellbore. In other embodiments, downhole tools according to the present disclosure may be used outside a wellbore or other downhole environment used for the exploration or production of natural resources. For instance, downhole tools of the present disclosure may be used in a borehole used for placement of utility lines. Accordingly, the terms “wellbore,” “borehole” and the like should not be interpreted to limit tools, systems, assemblies, or methods of the present disclosure to any particular industry, field, or environment.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers’ specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. An untethered well logging apparatus, comprising:
a body;
a buoyancy adjusting mechanism disposed in the body and configured to increase a buoyancy of the untethered well logging apparatus when a predetermined condition is met; and
a downhole sensor disposed with the body.

2. The untethered well logging apparatus of claim 1, wherein the downhole sensor comprises a depth sensor.

3. The untethered well logging apparatus of claim 1, wherein the downhole sensor comprises a low friction caliper device attached to the body and configured to determine a distance between the body and an internal wall of a well.

4. The untethered well logging apparatus of claim 3, wherein the low friction caliper device comprises:
a sensing arm configured to articulate relative to the body;
a spring urging the sensing arm radially outward relative to the body;
a sensor configured to determine an angle between the sensing arm and the body; and
a roller provided at a distal end of the sensing arm and configured to roll on the internal wall of the well.

5. The untethered well logging apparatus of claim 4, further comprising an actuator configured to retract the sensing arm relative to the body.

6. The untethered well logging apparatus of claim 5, wherein the body comprises a notch configured to receive the roller of the sensing arm when the sensing arm is retracted.

7. The untethered well logging apparatus of claim 3, wherein the low friction caliper device comprises:
a sensing finger configured to extend radially outward relative to the body;
a spring urging the sensing finger radially outward relative to the body;
a sensor arrangement configured to determine an outward radial position of the sensing finger; and
a bow spring having a central portion attached to a distal end of the sensing finger.

8. The untethered well logging apparatus of claim 7, wherein the bow spring comprises a first end and a second end slidably attached to the body.

9. The untethered well logging apparatus of claim 3, wherein the low friction caliper device comprises a piezoelectric sensor configured to generate an ultrasound wave and receive an ultrasound echo reflected by the internal wall.

10. The untethered well logging apparatus of claim 9, wherein the piezoelectric sensor is convex and configured to generate a pseudo-spherical ultrasound wave.

11. The untethered well logging apparatus of claim 9, wherein the downhole sensor comprises a fluid density sensor.

12. The untethered well logging apparatus of claim 11, wherein the fluid density sensor comprises a differential pressure sensor.

13. The untethered well logging apparatus of claim 3, wherein the low friction caliper device comprises:
a sphere configured to freely rotate and to move relative to the body;
an urging element urging the sphere radially outward relative to the body; and
a sensor assembly configured to determine a position of the sphere relative to the body.

14. A well logging apparatus, comprising:
a body having a main axis and an outer surface;
a buoyancy adjusting mechanism configured to increase a buoyancy of the well logging apparatus when a predetermined condition is met; and
a low friction caliper device provided on the outer surface of the body and configured to determine a distance between the body and an internal wall of a well.

15. The well logging apparatus of claim 14, wherein the low friction caliper device comprises:
a sensing arm configured to articulate relative to the body;
a spring urging the sensing arm radially outward relative to the body;
a sensor configured to determine an angle between the sensing arm and the body; and
a roller provided at a distal end of the sensing arm and configured to roll on the internal wall of the well.

16. The well logging apparatus of claim 15, further comprising an actuator configured to retract the sensing arm relative to the body.

17. An untethered well logging apparatus, comprising:
a body having a main axis and an outer surface;
a ballast tank having an inlet port provided in an upper portion of the ballast tank and an outlet port provided in a lower portion of the ballast tank, the outlet port being connected to an outside environment;
a high-pressure tank configured to store ballast gas in a compressed state;
a valve connecting the high-pressure tank with the inlet port of the ballast tank; and
a caliper device provided on the outer surface of the body and configured to determine a distance between the body and an internal wall of a well.

18. The untethered well logging apparatus of claim 17, wherein the caliper device comprises a sensing arm articulated with the body in a pivotable fashion, a spring urging the sensing arm towards an outward direction, and a roller provided at a distal end of the sensing arm and configured to roll on the internal wall of the well.

19. The untethered well logging apparatus of claim 17, wherein the caliper device comprises a piezoelectric sensor configured to generate an ultrasound wave and to receive an ultrasound echo reflected by the internal wall of the well.

20. The untethered well logging apparatus of claim 19, wherein the caliper device further comprises a differential pressure sensor configured to measure fluid density for determining sound velocity.
